(19) United States
(12) Patent Application Publication
(10) Pub. No.: US 2003/0229107 A1
(43) Pub. Date: Dec. 11, 2003

(54) Magnetic resonance with stimulation

(76) Inventors: Ronald Cowan, Brentwood, TN (US); Perry Renshaw, Bedford, MA (US); Blaise deB. Frederick, Watertown, MA (US); Scott Lukas, Boxborough, MA (US)

Correspondence Address: CHARLES H. SANDERS, Fish & Richardson P.C., 225 Franklin Street, Boston, MA, 02110-2804, US

(21) Appl. No.: 10/286,349

(22) Filed: Nov. 1, 2002

Related U.S. Application Data
(60) Provisional application No. 60/350,278, filed on Nov. 2, 2001.

(51) Int. Cl. A61K 31/522; A61N 1/30; A61K 31/445; A61K 49/00

(52) U.S. Cl. 514/263.33; 514/317; 514/649; 514/304; 604/20; 424/9.3

(57) ABSTRACT

This invention involves blood oxygen level dependent (BOLD) functional magnetic resonance imaging (fMRI) using external stimuli. Different colors of light can be used as the stimulus to assess differential BOLD response to diagnose neurological disorders (e.g., Parkinson's disease, attention deficit disorder, schizophrenia, and substance abuse). In addition, very low dose administration of a drug that affects intracerebral vasculature can enhance BOLD response, facilitating detection and improving diagnostic capabilities. Using a very low dose of d-amphetamine, BOLD response to blue light is increased.

### CROSS-REFERENCE TO RELATED APPLICATIONS

This application claims priority to U.S. Provisional Patent Application Serial No. 60/350,278, filed on Nov. 2, 2001.

### STATEMENT AS TO FEDERALLY SPONSORED RESEARCH

This invention was funded by grants DA09448, DA00366-01, and DA00343 from the National Institute on Drug Abuse. The government has certain rights in the invention.

### TECHNICAL FIELD

This invention relates to the use of magnetic resonance techniques with external stimuli.

### BACKGROUND

Disorders such as Parkinson's disease, attention deficit disorder, schizophrenia, substance abuse, and mood and anxiety disorders generally involve imbalances in neurotransmitters. These disorders are widespread and can be severely debilitating. One example of a neurotransmitter known to be associated with specific disorders is the catecholamine neurotransmitter dopamine. Dopamine is implicated in the pathophysiology of a variety of disease states such as Parkinson's disease, attention deficit disorder, schizophrenia, and substance abuse. This prominent neurotransmitter is found in the retina and is present at all levels of the visual pathway to primary visual cortex.

Visual stimuli are known to have effects on dopamine levels due to their presence in the visual pathway and the primary visual cortex. These neural effects can be measured indirectly using functional magnetic resonance imaging (fMRI). Functional magnetic resonance imaging is used to measure time-dependent changes in neural activity using a fast MR scan sequence, such as echoplanar imaging (EPI). This technique has been used to detect the effect of external stimuli on cortical activation. In particular, blood oxygen level dependent functional magnetic resonance imaging (BOLD fMRI) has been used to assess cortical function in humans in conjunction with visual stimuli. BOLD fMRI measures activity dependent increases in local blood flow, with resultant decreases in the local deoxyhemoglobin concentration, as a surrogate marker for increased local neuronal activity.

### SUMMARY

The invention features methods of fMRI using external stimuli. These external stimuli are both visual and pharmaceutical.

In one aspect, the present invention features a method of fMRI that measures BOLD responses of a subject to light having different wavelengths. The method involves applying a fMRI sequence to a subject, exposing the subject to photic stimulation using light having at least two wavelengths during the FMRI sequence, and processing data obtained during the functional magnetic resonance sequence to evaluate BOLD responses of the subject to the light. This method affords measurement of differential neural response to different colors of light, and aspects of these responses can be used to evaluate changes in neural conditions associated with neurological disorders.

Embodiments of this aspect can include one or more of the following features. The method can be applied to measure BOLD responses of human subjects to different wavelengths of light. In particular, the method can be applied to the human brain. Among the disorders that can be assessed using this method are Parkinson's disease, schizophrenia, or attention deficient disorder. In stimulating subjects with light, the photic stimulation can be alternated with periods without photic stimulation. This permits comparison of stimulated and unstimulated states. The different wavelengths of light can be red light and blue light, i.e., light having respective wavelengths of approximately 660 nm and 470 nm. The use of red and blue light is useful to assess the greater sensitivity of blue light responses to dopamine levels. Blue light response covaries with altered dopamine conditions such as schizophrenia, Parkinson's disease, and substance abuse. This permits use of BOLD response to blue light in comparison with red light to be used in diagnosing these central nervous system disorders of altered dopaminergic function.

In additional to providing visual stimuli, the method can also involve administering a drug to the subject. The drug can be methylphenidate, epinephrine, norepinephrine, ephedrine, levoephedrine, phenlyephrine, cocaine, albuterol, metaproterenol, terbutaline, dobutamine, caffeine, theophylline, theobromine, pentoxifylline, a nitric oxide antagonists, an endothelin antagonists, a bradykinin antagonists, a substance P antagonists, a vasoactive intestinal polypeptide antagonist, an angiotensin agonist, an atrial natriuretic hormone antagonist, a neuropeptide Y agonist, or a combination of one or more of these agents.

In another aspect, the invention features a method of enhancing contrast in fMRI by increasing BOLD response to a stimulus. This method involves administering a drug that enhances contrast by affecting intracerebral vasculature to a subject, in a dosage selected to minimize its effect on a central nervous system, applying a FMRI sequence to the subject while the drug is affecting intracerebral vasculature, stimulating the subject while the drug is affecting intracerebral vasculature, and processing data obtained during the functional magnetic resonance sequence to evaluate BOLD responses to the stimulus while the drug is affecting intracerebral vasculature. Enhancing contrast in fMRI is useful to detect the typically small changes in BOLD responses that are detected using this technique, and this increased sensitivity is useful for diagnostic purposes, and signal changes that were not detectable using other methods can be observed with this technique. In certain embodiments of this method, the drug is d-amphetamine and the dosage is less than about 3 mg. One possible dosage of d-amphetamine is 2.5 mg.

In yet another aspect, the invention features a method of magnetic resonance imaging to measure BOLD responses of a subject to light having different wavelengths by administering about 2.5 mg of d-amphetamine to the subject, applying a fMRI sequence to the subject, exposing the subject to photic stimulation using red and blue light during the fMRI sequence, and processing data obtained during the functional magnetic resonance sequence to evaluate BOLD responses of the subject to the red and blue light.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described here. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagrammatic representation of the experimental protocol.

FIG. 2 is a representative correlation-map display of BOLD signal change through axial oblique sections of visual cortex from a single subject who received drug.

FIG. 3 is a table of the percentage of the BOLD signal changes due to blue light stimulation for both drug and placebo conditions.

FIG. 4 is a graph of mean activation from right and left V1 from all subjects for blue light stimulation.

FIG. 5 is a table of the percentage of the BOLD signal changes due to red light stimulation for both drug and placebo conditions.

FIG. 6 is a graph of mean activation from right and left V1 from all subjects for red light stimulation.

### DETAILED DESCRIPTION

Experimental Protocol

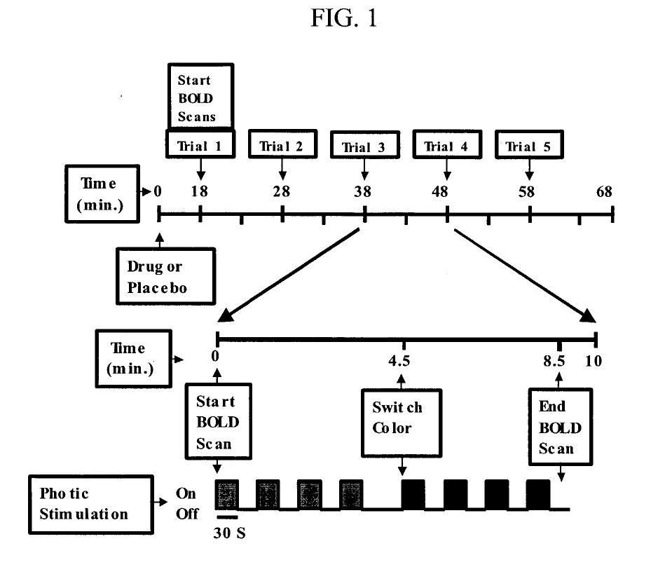

FIG. 1 is a diagrammatic representation of the photic stimulation and EPI protocol. The upper portion of the figure depicts the time course of the experiment. Drug or placebo was administered orally at the time defined as 0 minutes. Subjects received lactose placebo tablets or drug tablets (2.5 mg d-amphetamine sulfate (Dexedrine, SmithKline Beecham Pharmaceuticals, Philadelphia, Pa.)). The 2.5 mg tablet was prepared by halving a 5 mg tablet. D-amphetamine is converted into dopamine and passes into the brain. A very low dose of amphetamine was chosen to minimize the potential for amphetamine-induced movement.

Subjects were then placed in the scanner, goggles were fitted, and baseline structural imaging scans were performed. MR scans were performed on a 1.5 Tesla (T) Signa Echo-Speed (General Electric, Milwaukee, Wis.) whole body magnetic resonance scanner (level 5.8).

Anatomical brain imaging was performed on each subject in the sagittal, coronal and oblique axial planes to provide matched anatomical sections with detail for cross-referencing functional to anatomical images. Using a standard quadrature head coil, Ti-weighted volumetric 3D Spoiled Gradient Recall (SPGR) images were obtained with the following parameters: Flip angle=5 degrees, TR=300 ms, TE=9 ms, slice thickness=5 mm, field of view (FOV)=20×20 cm, matrix size 256×192 pixels for an in plane resolution of 0.95 mm.

Echo-planar imaging started at approximately 18 minutes and 5 trials were conducted. For BOLD imaging, gradient echo EPI axial images collected in an oblique plane parallel to the calcarine fissure were used to assess photic stimulation-induced BOLD signal changes. Three locations of 5 mm thickness with 0 mm skip were obtained to include the calcarine cortex and adjacent regions. Acquisition parameters were TR=2 s, flip angle=90 degrees, matrix=64×64 pixels, FOV=20×20 cm, 3×3 mm in-plane resolution. 256 images were obtained at each location using a 5 inch receive-only surface coil. During the BOLD acquisitions, periods of darkness alternated with periods of photic stimulation. Images were corrected for in-plane subject motion before analysis using the 2-dimensional Decoupled Automated Rotation and Translation (DART) registration algorithm, see Maas et al., Magnetic Resonance in Medicine, 37:131-139, 1997, which is incorporated by reference herein.

The middle of the diagram details a single trial. Within each trial, the start of the BOLD measurement (after discarding 4 dummy scans) was the zero time point. Each BOLD trial lasted 8 minutes 30 seconds, and images were collected at 2 s intervals. Red or blue photic stimulation started coincident with the onset of the BOLD trial (color order was varied, red was first in approximately half of all trials). The first four minutes of a trial consisted of a 4-minute epoch of red or blue photic stimulation. Each 4-minute epoch consisted of four 30 s periods of photic stimulation alternating with 4 30 s periods of darkness. An additional 30 s period of darkness was imposed during the color switch portion of the trial, and the 4-minute sequence of photic stimulation alternating with darkness was repeated for the second color. The total inter-scan time was approximately 10 minutes to account for post-imaging processing and storage. Individual time epochs varied more than is illustrated, and actual time measures were used in statistical analyses.

Color photic stimulation was delivered via a custom-designed set of stimulus goggles. The goggles have three sets of light emitting diodes (LEDs) that emit light at 470 nm (blue), 570 nm (green), and 660 nm (red), which can flash independently or in any combination. The optical wavelengths were chosen to match the response curves of the three color cones in the human eye; each frequency exciting one type of cone preferentially, see Gouras, Vision Research, 21:1591-1598, 1981. The LEDs and all control electronics were located outside of the magnet bore. The goggles were fed by a 20-foot long fiber optic bundle (South Coast Fiber Optics, Alachua Fla.). Each eye of the goggles had a 6-row by 8-column matrix of pixels.

Each color trial consisted of 8 Hz flash rate light delivered for 30 S on, 30 S off, 4 stimulus and 4 rest periods. Trials were repeated a total of 5 times (FIG. 1). Intensity was varied by changing flash duty cycle (within a 30 S stimulation period, all flashes were extremely short, 5 mS or less). Blue and red LED intensities were 0.12 lux as measured using a lux meter (Extech Instruments Foot Candle/Lux Meter, Waltham, Mass.) at the operational distance used for subject stimulation. For all experiments in this study, the entire 6 by 8 pixel array was flashed in each eye for each stimulus period against a dark background. Signal was analyzed for red and blue stimuli individually. All experiments were conducted in a darkened room.

Region of Interest (ROI) Determination

The ROI for this study was chosen in an attempt to study the physiologically most active sub-region (as defined by BOLD signal change) within right and left V1. The anatomical boundaries for V1 were determined as follows: The anterior boundary was the parieto-occipital sulcus. The posterior boundary was the occipital pole. The medial boundary was the interhemispheric fissure. The lateral boundary was determined for right and left sides as a maximum of 3 pixel widths lateral to the midline.

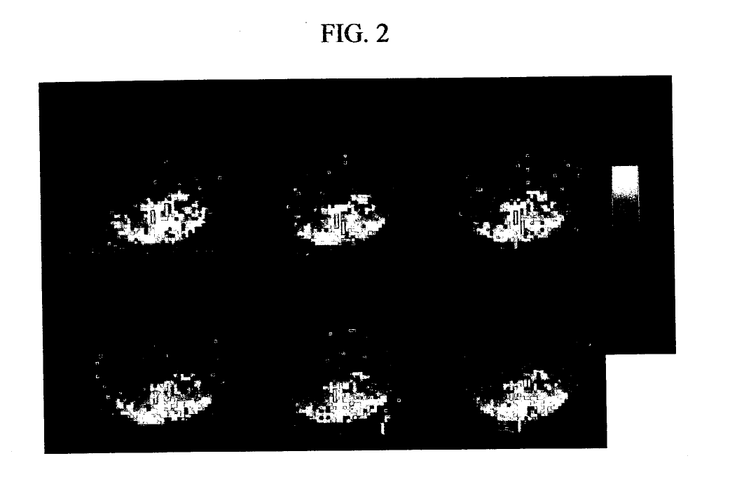

Once the anatomical boundaries for V1 were determined, a 1×4 contiguous pixel region whose long dimension was oriented parallel to the interhemispheric fissure was drawn within right V1 and left V1 to circumscribe a 4 pixel region having the highest correlation coefficient as determined by the correlation map display (see FIG. 2 for example of ROI). The magnitude of BOLD signal increase (activation) was determined as the percent change in the mean signal intensity within the ROI during the period of photic stimulation (average of signal from the 4 stimulation periods for each trial) compared to the mean baseline signal (average of signal from the 4 non-stimulus periods for each trial).

Image Analysis

Image analysis was performed using image analysis software. Specifically, an automated mapping procedure, see Mass et al., American Journal of Psychiatry, 155:124-126, 1998, which is incorporated by reference herein, was used to compare the correlation between the time course of signal change in a pixel with that of the time course of the photic stimulus according to the correlation coefficient detection method of Bandettini et al., Magnetic Resonance in Medicine, 30:161-173, 1993, which is incorporated by reference herein. Pixels whose correlation coefficient was above an arbitrarily chosen threshold (r=0.25) were identified and displayed using pseudo-color overlays of stepped changes in correlation.

FIG. 2 is a representative correlation-map display of BOLD signal change through axial oblique sections of visual cortex from a single subject who received drug. Green rectangles represent the ROI from right and left V1, which is placed in maximally activated 1×4 pixel region. The top row is subject response to fixed intensity blue light stimulation over time following ingestion of 2.5 mg d-amphetamine. The bottom row is same subject showing response to red light in the same experiment. Time post-drug increases from left to right. The calibration bar displays pseudo-color overlay scale as corresponding to correlation coefficient.

Statistical Analysis

Statistical analysis was performed using Stata, Version 6.0 (Stata Corporation, College Station, Tex.). Wald Chi² analysis with 1 degree of freedom was used to determine if there was an effect of drug on the mean activation across an average of all trials for each color (i.e. to compare red placebo and blue placebo means, red placebo means to red drug means, and blue placebo means to blue drug means). Linear regression modeling with robust estimation of standard errors was used to examine the effects of age and sex on BOLD signal change, and to determine if there was an effect of time following drug (time points were entered as the time of onset of a particular color trial following drug ingestion) on the mean signal change from the 1×4 region in right and left V1. Model fit was assessed using partial residual plot methods. An alpha level of 0.05 was used as the criterion for statistical significance for all tests.

### EXAMPLE

Fifteen volunteers (5 male; 10 female) participated in 22 functional MRI scans. Seven subjects participated in both placebo and drug administration studies; 8 subjects participated in either placebo or drug administration studies. To determine if iso-intense stimuli of blue and red light at 0.12 lux were equally effective at eliciting V1 BOLD signal change, the mean BOLD signal change in V1 was compared across all trials for combined right and left V1 for each color in the placebo condition. The mean BOLD signal increase to red light stimulation at 0.12 lux was 0.95% (S.D. 0.76). The mean BOLD signal increase to blue light stimulation at 0.12 lux, conversely, was 1.60% (S.D. 0.74). Wald Chi² comparison of the mean activations revealed a significant difference in the baseline (placebo condition) response to the two colors, with blue light eliciting a 68% greater BOLD signal increase than the corresponding intensity of red light (Wald Chi²=52.00, P<0.0001). The mean BOLD signal change from the lx4 ROI in right and left V1 was grouped across all trials for the drug and placebo condition to determine if damphetamine administration altered the mean BOLD signal change.

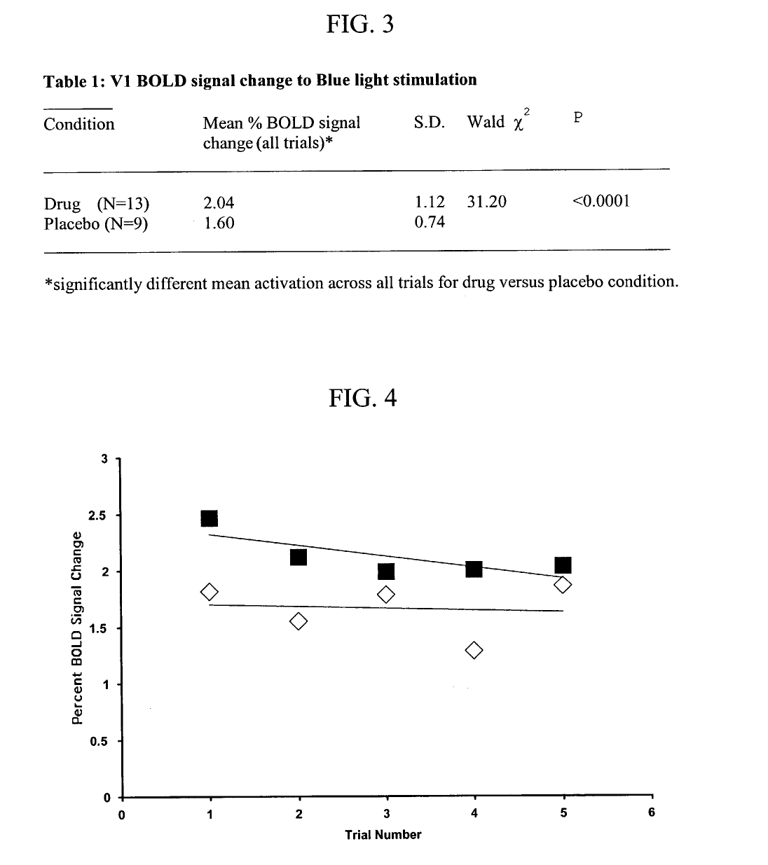

For blue light, the overall mean was significantly different in the Wald Chi² analysis, with the mean BOLD signal change being 28% higher in the drug (2.04%) condition than in the placebo (1.60%) condition. FIG. 3 is a table of the percentage of the BOLD signal changes due to blue light stimulation for both drug and placebo conditions.

To further characterize the time course of the effect of drug on the blue light response, grouped means were plotted as percent BOLD signal change versus trial number for the blue light placebo and drug conditions (FIG. 4). FIG. 4 is a graph of mean activation from right and left V1 from all subjects for blue light stimulation. Y axis is percent signal change from baseline. X axis is trial number. Solid squares represent data from the subjects who received drug, hollow diamonds represent data from the subjects who received placebo. Lines are least squares regression lines. Because drug levels were expected to increase over time during the experiment, a linear regression analysis was used to determine if there was an effect of time following drug administration on the BOLD signal response to blue light. For the drug condition, there was a significant effect of time following drug administration on BOLD signal change (Z=−2.471, P=0.013) with decreasing BOLD signal over time. For the placebo condition, there was no significant effect of time on BOLD signal change (Z=−0.355, P=0.722).

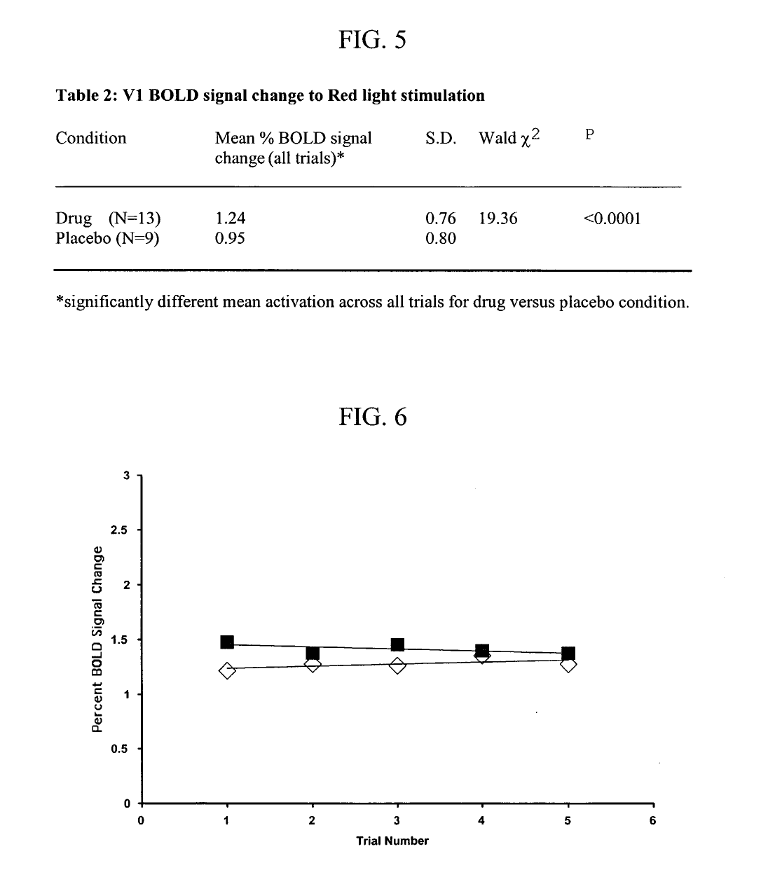

For red light, the overall mean was also significantly different in the Wald Chi² analysis, with the mean BOLD signal change being 30% higher in the drug (1.24%) condition than in the placebo (0.95%) condition. FIG. 5 is a table of the percentage of the BOLD signal changes due to red light stimulation for both drug and placebo conditions. To further characterize the time course of the effect of drug on the red light response, grouped means were plotted as percent BOLD signal change versus trial number for the red light placebo and drug conditions (FIG. 6). FIG. 6 is a graph of mean activation from right and left V1 from all subjects for red light stimulation. Y axis is percent signal change from baseline. X axis is trial number. Solid squares represent data from the subjects who received drug, hollow diamonds represent data from the subjects who received placebo. Lines are least squares regression lines. When linear regression analysis was used to determine if there was an effect of time following drug administration on the BOLD signal response to red light, there was no significant effect of drug over time (Z=0.470, P=0.638). Similarly, there was no effect of time on BOLD signal response following placebo administration (Z=−0.880, P=0.379).

These results demonstrate that the overall BOLD response to red and blue light is enhanced by very low dose d-amphetamine administration, and the blue light response shows a differential sensitivity to drug administration. Specifically, the mean photic stimulation elicited BOLD signal intensity changes across all trials were higher for blue and for red light in the drug versus the placebo condition. Secondly, there was no effect of time on the red light elicited BOLD signal, but there was a clear time-related peak and decline in blue light evoked BOLD signal over time.

The sustained blue light response appears to be part of a common augmentation of the Vi BOLD response to both red and blue light that persists throughout all trials By contrast, the transient augmentation of the V1 BOLD response to blue light is largely neuronal in origin. The data indicate that at early time points following drug administration, there is a large enhancement of the BOLD response to blue light, that decays over time, yielding to a persistent lower-level augmentation. Blue light response is more directly influenced by changes in amphetamine-induced dopamine release than is the red light response.

This study also demonstrates that very low dose amphetamine administration produces a general increase in BOLD response to a fixed stimulus. This effect can be exploited to enhance fMRI signal to detect regional BOLD signal changes that were previously below the level of detectability at baseline for a wide variety of experimental paradigms. A very low dose of amphetamine avoids confounding the neural response to non-pharmaceutical stimuli, such as visual stimuli, with nervous system response to a pharmaceutical stimulus. However, this very low dose of d-amphetamine still has an effect on the intracerebral vasculature. Since BOLD response is dependent on local intracerebral vasculature, this very low dose affords improved BOLD response. Medications with similar effects on the vasculature will likewise provide enhanced contrast without significant nervous system effects if administered in very low doses.

A number of embodiments of the invention have been described. Nevertheless, it will be understood that various modifications may be made without departing from the spirit and scope of the invention. Accordingly, other embodiments are within the scope of the following claims.

What is claimed is:

1. A method comprising:
(a) applying a functional magnetic resonance imaging sequence to a subject;
(b) exposing the subject to photic stimulation using light having at least two wavelengths during the functional magnetic resonance imaging sequence; and
(c) processing data obtained during the functional magnetic resonance sequence to evaluate blood oxygen level dependent responses of the subject to the at least two wavelengths of light.

2. The method of claim 1 wherein the subject is a human.

3. The method of claim 1 wherein the subject is a human brain.

4. The method of claim 1 further comprising
(d) using the evaluation of blood oxygen level responses to diagnose whether the subject has Parkinson's disease, schizophrenia, or attention deficient disorder.

5. The method of claim 1 wherein the photic stimulation is alternated with periods without photic stimulation during the functional magnetic resonance sequence.

6. The method of claim 1 wherein the light having at least two wavelengths comprises red light and blue light.

7. The method of claim 1 wherein the at least two wavelengths are approximately 470 nm and 660 nm.

8. The method of claim 1 further comprising
(d) administering a drug to the subject.

9. The method of claim 8 wherein the drug is methylphenidate, epinephrine, norepinephrine, ephedrine, levoephedrine, phenlyephrine, cocaine, albuterol, metaproterenol, terbutaline, dobutamine, caffeine, theophylline, theobromine, pentoxifylline, a nitric oxide antagonists, an endothelin antagonists, a bradykinin antagonists, a substance P antagonists, a vasoactive intestinal polypeptide antagonist, an angiotensin agonist, an atrial natriuretic hormone antagonist, a neuropeptide Y agonist, or a combination of one or more of these agents.

10. A method of enhancing contrast in functional magnetic resonance imaging by increasing blood oxygen level dependent response to a stimulus, the method comprising:
(a) administering a drug that enhances contrast by affecting intracerebral vasculature to a subject, in a dosage selected to minimize its effect on a central nervous system;
(b) applying a functional magnetic resonance imaging sequence to the subject while the drug is affecting intracerebral vasculature;
(c) stimulating the subject while the drug is affecting intracerebral vasculature; and
(d) processing data obtained during the functional magnetic resonance sequence to evaluate blood oxygen level dependent responses to the stimulus while the drug is affecting intracerebral vasculature.

11. The method of claim 10 wherein the subject is a human brain.

12. The method of claim 10 further comprising
(d) diagnosing whether the subject has Parkinson's disease, schizophrenia, or attention deficient disorder.

13. The method of claim 10 wherein the stimulus is photic stimulation.

14. The method of claim 13 wherein the photic stimulation is alternated with periods without photic stimulation.

15. The method of claim 13 wherein the photic stimulation comprises light having at least two wavelengths.

16. The method of claim 15 wherein the light having at least two wavelengths comprises red light and blue light.

17. The method of claim 10 wherein the at least two wavelengths are approximately 470 nm and 660 nm.

18. The method of claim 10 wherein the drug is methylphenidate, epinephrine, norepinephrine, ephedrine, levoephedrine, phenlyephrine, cocaine, albuterol, metaproterenol, terbutaline, dobutamine, caffeine, theophylline, theobromine, pentoxifylline, a nitric oxide antagonists, an endothelin antagonists, a bradykinin antagonists, a substance P antagonists, a vasoactive intestinal polypeptide antagonist, an angiotensin agonist, an atrial natriuretic hormone antagonist, a neuropeptide Y agonist, or a combination of one or more of these agents.

19. The method of claim 10 wherein the drug is d-amphetamine and the dosage is less than about 3 mg.

20. The method of claim 19 wherein the dosage is about 2.5 mg.

21. A method of magnetic resonance imaging to measure blood oxygen level dependent responses of a subject to light having at least two wavelengths:
(a) administering about 2.5 mg of d-amphetamine to the subject;
(b) applying a functional magnetic resonance imaging sequence to the subject;
(a) exposing the subject to photic stimulation using red and blue light during the functional magnetic resonance imaging sequence; and
(d) processing data obtained during the functional magnetic resonance sequence to evaluate blood oxygen level dependent responses of the subject to the red and blue light.
